(19) United States
(12) Patent Application Publication
(10) Pub. No.: US 2023/0149005 A1
(43) Pub. Date: May 18, 2023

(54) SYSTEMS AND METHODS FOR CLOSURE OF TISSUE OPENINGS

(71) Applicant: XDOT MEDICAL INC., Maple Grove, MN (US)

(72) Inventor: Aashiish AGNIHOTRI, Maple Grove, MN (US)

(21) Appl. No.: 18/054,777

(22) Filed: Nov. 11, 2022

Related U.S. Application Data
(60) Provisional application No. 63/278,552, filed on Nov. 12, 2021.

(51) Int. Cl. A61B 17/00; A61B 17/04

(52) U.S. Cl. CPC A61B 17/0057; A61B 17/0401

(57) ABSTRACT

The present disclosure relates to a system for radial cinching of an opening in an internal biological structure. The system includes, a tensioning tube, a plurality of sutures disposed within the tensioning tube, a plurality of delivery tubes. Each delivery tube is configured to receive a distal end of one of the plurality of sutures. The plurality of delivery tubes are further configured to deploy the distal ends of the plurality of sutures proximate a periphery of the opening. Upon deployment of the sutures proximate the periphery of the opening, one or more of the plurality of sutures are cinched while movement of portions of the plurality of sutures are constrained by the tensioning tube causes radial cinching of the periphery to close the opening.

### CLAIM OF PRIORITY

This application claims priority to U.S. Provisional Appl. No. 63/278,552 filed on Nov. 12, 2021, the disclosure of which is incorporated herein by reference in its entirety.

### FIELD OF THE DISCLOSURE

The present invention relates generally to systems and methods for closing openings in biological structure, and more particularly to suture-based systems and methods for closing openings.

### BACKGROUND OF THE DISCLOSURE

By nature of their location, the closure of openings in biological structures, particularly internal biological structures, is inherently difficult. For example, holes, defects, or cavities (collectively, “openings”) may exist within a body naturally, congenitally, or created by a procedure or device. For instance, a patent foramen ovale (“PFO”) is a serious septal defect that can occur between the left and right atria of the heart. Similarly, a patent ductus arteriosus (“PDA”) is an abnormal shunt between the aorta and pulmonary artery. Still other septal defects can occur between the various chambers of the heart, such as atrial-septal defects (ASD's), ventricular-septal defects (VSD's), and the like. In yet other cases, left atrial appendage, which is an organ in the left atrium of a human heart, causes issues such as, thrombus, and strokes, and may need to be closed to prevent such issues. The openings can vary in size and in some cases, requires surgery to ligate and/or close the opening.

In recent years, there has been a shift towards closing such openings by routing a device into a body using a delivery catheter or similar device. For example, the delivery catheter can be routed through the transfemoral route to a heart or cardiovascular system and a device can be deployed through the delivery catheter to close an opening. There are many advantages to closing holes or defects in this method including: fast recovery and low risks of complications as compared more invasive surgeries such as open-heart surgery or other more invasive procedures traditionally required to close openings in internal tissue. However, current solutions for closing openings within biological structures often preclude future interventions. Other solutions are highly complex and can leave foreign bodies within a heart or a human body.

The current disclosure describes devices and methods directed towards solving some of the issues discussed above.

### SUMMARY OF THE DISCLOSURE

The present disclosure relates to systems, methods, and devices for radial cinching of an opening in an internal biological structure.

In some aspects, the disclosure relates to a system for radial cinching of an opening in an internal biological structure, including a tensioning tube that extends between a distal end and a proximal end. The system may additionally include a plurality of sutures that are configured for deployment within the tensioning tube from the proximal end to exit via the distal end of the tensioning tube. The tensioning tube may be configured to constrain movement of portions of the plurality of sutures disposed within the tensioning tube away from each other. The system additionally may include a plurality of delivery tubes, each of the plurality of delivery tubes are configured to receive a distal end of one the plurality of sutures after exiting from the distal end of the tensioning tube. The plurality of delivery tubes may further be configured to deploy the distal ends of the plurality of sutures proximate a periphery of the opening. Upon deployment of the sutures proximate the periphery of the opening, of one or more of the plurality of sutures can be tensioned while movement of portions of the plurality of sutures is constrained by the tensioning tube which causes radial cinching of the periphery to close the opening.

In some examples, the system may further include a delivery catheter, and the tensioning tube and the plurality of tubes are deployable within a lumen of the delivery catheter. In some embodiments, the system additionally may include a guidewire. Optionally, the system may include a stabilizer that is deployable over the guidewire and configured to be disposed on a side of the opening that does not include the plurality of sutures such that the stabilizer can stabilize the plurality of tubes with respect to the opening during deployment of the sutures. In some embodiments, the stabilizer is selected from at least one of the following: a balloon, a stent, a disc, or a sheath. Additionally and/or alternatively, a positioning element deployable over the guidewire may be included in the system. The positioning element is configured to control positioning of the sutures during deployment by causing outward bending of one or more of the plurality of delivery tubes. The positioning element is selected from at least one of the following: a balloon, a stent, a disc, or a sheath.

In some examples, the plurality of sutures are configured to be either symmetrically deployed or asymmetrically deployed around the periphery of the opening.

In some other examples, the system may also include a suture lock configured to lock the plurality of sutures in a tensioned state after the opening is closed.

In some examples, the distal ends of each of the plurality of sutures may be associated with a suture anchor, the suture anchor being in a compressed state when the distal end of the corresponding suture is disposed within a delivery tube. In some examples, the suture anchor is configured to be in an expanded state upon deployment from the delivery tube and anchor the corresponding suture to the periphery.

In some examples, each of the plurality of sutures may be configured to be individually controlled for deployment and tensioning.

In some examples, the opening may be located within a cardiovascular region.

In some examples, the sutures are made of a material that is absorbable by a human body.

In some other aspects, the disclosure relates to a system for radial cinching of an opening in an internal biological structure. The system includes: a guidewire, a delivery catheter, and a suturing device. In some examples the suturing device may include an axially rigid tensioning tube that is deployable, via the delivery catheter, to the opening. The suturing device additionally may include a plurality of sutures configured for deployment within the tensioning tube from a proximal end of the tensioning tube to exit via a distal end of the tensioning tube. The tensioning tube may be configured to constrain movement of portions of the plurality of sutures disposed within the tensioning tube away from each other. The suturing device may additionally include a plurality of delivery tubes disposed within the delivery catheter. Each of the plurality of delivery tubes may be configured to receive a distal end of one the plurality of sutures after exiting from the distal end of the tensioning tube. The plurality of delivery tubes may further be configured to deploy the distal ends of the plurality of sutures proximate a periphery of the opening. The distal ends of the plurality of sutures can be associated with suture anchors. A suture lock may, optionally, be disposed near the distal end of the tensioning tube. Upon deployment of the sutures proximate the periphery of the opening, tensioning of one or more of the plurality of sutures while movement of portions of the plurality of sutures is constrained by the tensioning tube causes radial cinching of the periphery to close the opening.

In some examples, each of the plurality of delivery tubes may include a slot that extends, from a distal end thereof, at least partially along a length of that delivery tube, the slot being configured to receive the distal end of that one the plurality of sutures after exiting from the distal end of the tensioning tube.

Optionally, each of the plurality of delivery tubes may include a mechanism for deployment, by pushing, of a suture from a distal end of that delivery tube.

In various embodiments, the suture lock may be configured to be advanced over the plurality of sutures and to lock the plurality of sutures in a tensioned state.

Optionally, the system may include a stabilizer that is deployable over the guidewire and configured to be disposed on a side of the opening that does not include the plurality of sutures such that the stabilizer can stabilize the plurality of tubes with respect to the opening during deployment of the sutures.

In some other aspects, the present disclosure relates to a method of closing an opening within an internal biological structure. The method may include the steps of: deploying a delivery catheter over a guidewire towards the opening; deploying a tensioning tube within a lumen of the delivery catheter, the tensioning tube being axially rigid; deploying a plurality of sutures through a proximal end of the tensioning tube to exit from a distal end of the tensioning tube, the plurality of sutures each including a suture anchor at a distal end of the suture; deploying a plurality of delivery tubes through the lumen of the delivery catheter, distal ends of the each of the plurality of sutures being disposed within one of the plurality of delivery tubes such that the suture anchors are in a compressed state; anchoring, using the suture anchors, the sutures into peripheral tissue around the opening by pushing the sutures from distal ends of each of the plurality of delivery tubes; and tensioning the plurality of sutures while movement of portions of the plurality of sutures is constrained by the tensioning tube to cause radial cinching of the periphery to close the opening.

In various embodiments, the methods may also include deploying a suture lock and activating the suture lock for locking the sutures in a tensioned state after radial cinching of the periphery.

In various embodiments, the methods may also include deploying an expandable stabilizing device through the lumen of the delivery catheter for anchoring the tensioning tube and the plurality of delivery tubes with respect to the opening. Optionally, the expandable stabilizing device may be expanded after deploying the stabilizing device.

In various embodiments, the methods may also include deploying a positioning element through the lumen of the delivery catheter, and causing, using the positioning element, outward bending of the plurality of delivery tubes for controlling a position of one or more of the suture anchors.

Optionally, the delivery catheter and the plurality of delivery tubes may be withdrawn before tensioning the plurality of sutures. Additionally and/or alternatively, the tensioning tube and the guidewire may be withdrawn after tensioning the plurality of sutures.

In some other embodiments, the present disclosure relates to a method of performing a procedure within an internal biological structure. The method may include the steps of deploying a delivery catheter over a guidewire towards the internal biological structure; deploying a tensioning tube within a lumen of the delivery catheter, the tensioning tube being axially rigid; deploying a plurality of sutures through a proximal end of the tensioning tube to exit from a distal end of the tensioning tube, the plurality of sutures each including a suture anchor at a distal end of the suture; deploying a plurality of delivery tubes through the lumen of the delivery catheter, distal ends of the each of the plurality of sutures being disposed within one of the plurality of delivery tubes such that the suture anchors are in a compressed state; anchoring, using the suture anchors, the sutures into peripheral tissue around the opening by pushing the sutures from distal ends of each of the plurality of delivery tubes; withdrawing the delivery catheter; deploying a procedure tool for creating an opening in an area between the anchored sutures; performing, via the opening, the procedure; and tensioning the plurality of sutures while movement of portions of the plurality of sutures is constrained by the tensioning tube to cause radial cinching of the periphery to close the opening.

In some examples, the methods may include withdrawing the procedure tool before tensioning the plurality of sutures. Optionally, tensioning the sutures may include tensioning the sutures during performing of the procedure to keep the opening at least partially cinched over the procedure tool.

### BRIEF DESCRIPTION OF THE DRAWINGS

FIG. 1A is a schematic illustrating a perspective view of a system for deploying sutures for closing an opening in accordance with the principles of the present disclosure.

FIG. 1B is a schematic illustrating a cross-sectional view of the system of FIG. 1A from a distal end thereof, in accordance with the principles of the present disclosure.

FIGS. 2A and 2B illustrates an example positioning element.

FIG. 3A-3E are suture anchors in accordance with the principles of the present disclosure for use with the system of FIG. 2.

FIG. 4A is a flowchart outlining an example method of using the system of FIGS. 1A and 1B (collectively, FIG. 1).

FIG. 4B is a flowchart outlining an example method of using the system of FIG. 1.

FIG. 5 is a short axis view of the heart at the level of the right atrium (RA) and the left atrium (LA), in a plane generally parallel to the atrio-ventricular groove, and at the level of the aortic valve, showing a PFO track.

FIGS. 6A-6H illustrate closure of a PFO track opening using the system of FIG. 1.

FIGS. 7A-7E illustrate closure of a base of a left atrial appendage using the system of FIG. 1.

FIG. 8 is an illustration of sutures deployed for trans-delivery catheter-edge-to-edge repair using the system of FIG. 1.

FIGS. 9A-9E illustrate closure of a large opening using sutures and the system of FIG. 1.

FIGS. 10A-10I illustrate examples of suture and suture anchor placements about an opening for radial cinching closure of the opening.

### DETAILED DESCRIPTION

The devices and methods of the present disclosure may be understood more readily by reference to the following detailed description of the embodiments taken in connection with the accompanying drawing figures, which form a part of this disclosure. It is to be understood that this application is not limited to the specific devices, methods, conditions, or parameters described and/or shown herein, and that the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, proximal, distal, horizontal, vertical, top, upper, lower, bottom, left and right, are for illustrative purposes only and can be varied within the scope of the disclosure. For example, the references “upper” and “lower” are relative and used only in the context to the other and are not necessarily “superior” and “inferior”. While the examples provided in this disclosure generally relate to closure of holes, defects, or cavities in the cardiovascular system, disclosed systems and methods may be used for closure of holes, defects or cavities, such as, but not limited to, in various holes, openings and/or defects in biological tissue within a subject in vivo.

As used herein, the term “proximal” means closest to the operator (less into the body) and “distal” means furthest from the operator (further into the body). In positioning a medical device from a downstream access point, distal is more upstream and proximal is more downstream.

The term “suture” as used herein can be monofilament or multifilament elongate flexible tensioning devices made of flexible or non-flexible material with sufficient tensile strength for providing a desired force. The suture can be a strand, a wire, a cord, a fiber, a yarn, a filament, a cable, a thread, or the like, and these terms may be used interchangeably. Suture materials may generally be broken into two categories, absorbable (capable of being broken down and absorbed into the body) and non-absorbable (should be manually removed from the body). Example materials include but are not limited to polyglycolic acid, polylactic acid (PLA), polypropylene, polyester, silicone, polyurethane, stainless steel, nickel titanium alloy, nylon, Kevlar fiber, or others.

As used herein, the terms “closure” “closed”, “closing” refer to reducing the size (e.g., diameter, area, volume, etc.) of an opening in a biological structure from an initial size to a smaller size.

Access to an opening in an internal biological structure through invasive surgery introduces a high level of risk that can result in serious complications for a subject. Access to an opening remotely with a delivery catheter or equivalent device is less risky, but treatment or closure of the opening itself is made more difficult given the limited physical abilities of the delivery catheter. Furthermore, as discussed above, existing devices for closure of openings in internal biological structures preclude future interventions (e.g., septal closure devices preclude future interventions via a catheter in the left atrium of the heart), are technically complex to deploy, may leave large foreign body inside the body, and/or are not adaptable to patient specific anatomies. In some cases, if the suture(s) are deployed through a delivery catheter, it can be difficult to cinch the sutures and close the opening due the suture(s) typically being pulled outwardly away from the location the suture is anchored into rather than radially inward (e.g., towards the center of the opening).

In the various embodiments, the devices, methods, and systems of the present disclosure provide closure of openings in internal biological structures, whether formed by trauma, surgery, disease, normal anatomy, or other means, by radial cinching and may overcome at least some of the issues discussed above. Specifically, embodiments of the current disclosure describe closure devices and methods used to apply sutures and/or suture closed openings at, within, or into a biological structure. Furthermore, sutures are pulled inwardly towards the opening to close the opening by inward cinching of a periphery of the opening. This prevents outward pulling on, for example, tissue (or structures) within which the suture anchors are deployed, further widening of the opening and/or sub-optimal closing. To accomplish this, in some examples, the sutures are deployed through a tensioning tube that prevents outwards movement of the sutures while being tensioned. Furthermore, delivery tubes are provided for deploying and anchoring distal portions of the sutures (e.g., via suture anchors) around the periphery of an opening. However, it shall be understood that the disclosure herein is not limited to merely solving these specific problems. Additionally, while the devices and techniques disclosed herein are described with respect to a human body or patient, it is understood that the devices and techniques may in suitable circumstances be applied to a non-human patient (i.e., in veterinary medicine).

Optionally, the suturing devices and methods can be used to place sutures prior to performing a surgical procedure proximate to an opening or access to be created during the surgical procedure (i.e., to prepare access for the procedure). The placed sutures can then be used to radially cinch an opening after any devices or tools used during the procedure are withdrawn, closing the opening while the surgical device or tool leaves the opening. Optionally, the sutures may also be used to tighten (by radial cinching) the periphery of the opening around a device or tool while the device or tool is inside the opening during the course of the procedure.

Additionally and/or alternatively the devices and methods of the current disclosure allow for future interventions that require creation of an opening in the same biological structure or tissue. Specifically, the sutures, suture anchors, and/or the suture lock left behind after closure of the opening have a much smaller footprint compared to prior art closure device (e.g., a coin sized mesh) allowing for space for creation of a new opening. The smaller footprint is further reduced as the sutures, suture anchors, and/or the suture lock are optionally absorbed over time. A small footprint further allows for tissue to heal at the site of the opening of the device of the current disclosure. Given their larger footprint, prior art closure devices are also associated with a higher risk of embolization, tissue erosion, or other complications; which may be reduced using the principles of this disclosure.

Embodiments described herein can, therefore, be used to repeatedly provide access to (via an opening) and/or suture an opening to a biological structure where space inside of the biological structure is limited or where the biological structure cannot be accessed without forming an opening.

In some examples, sutures are anchored by deployment of suture anchors (via delivery tubes) at two or more locations about the periphery of an opening in a biological structure. After being distally anchored, sutures extend through a tensioning tube such that proximal ends of the sutures can be maneuvered after exiting a proximal end of the tensioning tube. When the sutures are tensioned, they cause radially inward cinching of the peripheral tissue of the opening because the tensioning tube (which has a distal end positioned near near the opening) prevents outward radial forces and prevents outwards movement of the sutures, thereby closing the opening. In some examples, the delivery tubes and/or the tensioning tube are axially rigid (while being flexible for bending for, for example, transcatheter deployment within a delivery catheter) to prevent collapsing or kinking of the tubes in a direction perpendicular to the longitudinal axis of the tube. Each of a plurality of sutures connected to the suture anchors may be selectively and individually tensioned. In various embodiments, the arrangement of suture (once deployed around a vascular opening) may be configured to enable radial cinching by pulling the sutures (example, via suture anchors) towards a center of the suture arrangement where tissue opening is located. For example, the suture may be deployed into tissue and/or structures around the opening such that when the sutures are tensioned the sutures radially cinch the opening inwards by pulling on the tissue and/or structures. Similarly, the sutures may be deployed in a manner that they form approximate vertices of a square, triangle, pentagon, hexagon, or the like with the opening being between the arrangement (center or off center). Additionally, sutures may be deployed asymmetrically about an opening depending on the size and shape of the opening. Other arrangements are within the scope of this disclosure such as to allow for linear cinching, or the like.

A suture lock may, optionally, be used to lock the cinching sutures together in a tensioned state once the opening is closed.

Access to the opening to be closed will preferably be accomplished through the patient's vasculature in a “percutaneous” manner. By “percutaneous” it is meant that a location of the vasculature remote from the opening is accessed through the skin, typically using a surgical cut down procedure or a minimally invasive procedure, such as using needle access through, for example, the Seldinger technique. The ability to percutaneously access the remote vasculature is well-known and described in the patent and medical literature. Once percutaneous access is achieved, the interventional tools (e.g., the devices of this disclosure) and supporting delivery catheter(s) will be advanced to the opening intravascularly where they may be positioned adjacent the target opening in a variety of manners, as described elsewhere herein. However, it is understood that the devices and methods described herein can also be used under direct visualization (e.g., during open heart surgery) by the user or with indirect visualization using, for example, a surgical endoscope. For example, in non-cardiovascular procedures, access may be achieved endoscopically (e.g., using a medical imaging device). Other minimally invasive non-vascular approaches also may be performed.

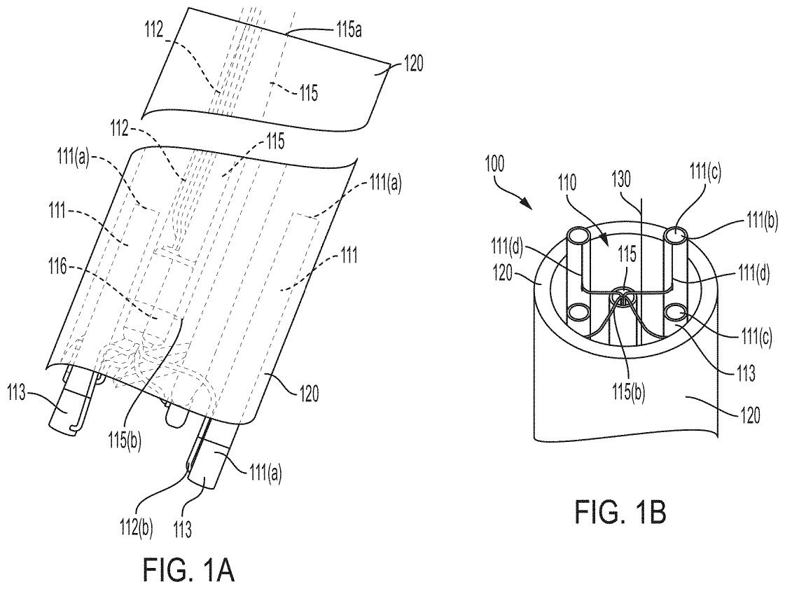

Referring now to FIGS. 1A and 1B, a system 100 in accordance with the principles of the present disclosure is shown. The system 100 may include a device 110 and a delivery catheter 120 for trans-catheter closure of openings within a biological structure such as an opening in a PFO track discussed below. It should be noted that the use of a delivery catheter is exemplary and the device 110 may be used/deployed without a delivery catheter (e.g., directly over a guidewire and/or within a sheath).

When a delivery catheter is used, the device 110 may be sized to be disposed within the delivery catheter 120 being utilized, and may be deployable via the delivery catheter. Any now or hereafter known delivery catheters may be used. In certain embodiments, the delivery catheter may be deployed proximate the opening to be closed over a guidewire 130, where the guidewire is itself positioned using any now or hereafter known techniques. For example, a cannula of an instrument, such as an angiographic needle, is inserted through the skin into an artery, such as the femoral artery, at the situs for the instrument's insertion. The needle cannula is held in place and a flexible guidewire is then passed through the cannula longitudinally into the artery until it reaches a desired depth. Once the guidewire is in place, the needle cannula is removed, leaving the guidewire in place. The system (e.g., delivery catheter 120 and/or device 110) is then advanced over the guidewire to the desired location, e.g., the opening. It should be noted that a sheath may be used instead of a delivery catheter.

The delivery catheter affords subsequent access to permit introduction of the device 110 (and/or other components of the system 100). In some embodiments, the delivery catheter 120 has a diameter of about 9 French to about 14 French, about 10-13 French, about 11-12 French, or the like. However, the disclosure is not limited to this range of sizes of interventional devices. In some embodiments, other sizes of interventional devices may be used. For example, devices of certain embodiments may work with size ranges of devices ranging from 12-24 French, 6-12 French, 9-24 French, as well as larger or smaller sizes. Size of the delivery catheter may depend on the number of sutures, size of opening, or the like.

The device 110 includes an elongated tubular member 115 (or “tensioning tube”) within which a plurality of sutures 112 are disposed and a plurality of delivery tubes 111. The system 110 and/or the device 110 may include other components such as positioning elements, stabilizers, suture anchors, or the like (discussed below). The tubular member may have a circular cross-section, a square cross-section, or the like.

The tensioning may extend between a proximal end 115(a) and a distal end 115(b) and is movable within a lumen of the delivery catheter 120 from a proximal end to a distal end thereof. The tensioning tube 115 may be advanced within the delivery catheter 120 (with or without a guidewire) such that the distal end 111(b) of each tube may be positioned against or near a tissue region surrounding an opening to be closed. The tensioning tube 115 is axially rigid such that when the distal end 115(b) is positioned against or near tissue and/or structure proximate the opening, it is configured to prevent or constrain movement of the sutures away from each other (i.e., outward movement of portions of the sutures that reside within the tensioning tube with respect to each other is constrained within the diameter of the tensioning tube). As such, when the anchored sutures are tensioned the sutures (and, hence, the peripheral tissue) are cinched radially inwards towards the opening, while the tensioning tube prevents outward pulling of the sutures and/or outward pulling of the peripheral tissue (i.e., away from the opening or towards the proximate end of the sutures). In some embodiments, the tensioning tube has a diameter of between 4-10 French, about 5-9 French, about 6-8 French, or the like.

The device 110 further includes a plurality of elongated tubular members (or “delivery tubes”) 111 spaced within a lumen of the delivery catheter 120 to radially surround the tensioning tube 115, and that are moveable along or through the delivery catheter 120. The tubular member 111 may have a circular cross-section, a square cross-section, or the like. Each of the delivery tubes 111 may extend between a distal end 111(b) and a proximal end 111(a), and may include a lumen 111(c) extending between the distal end and the proximal end. Optionally, each of the delivery tubes 111 includes a longitudinal slot 111(d) extending from the corresponding distal end along the length of that delivery tube (the slot may not extend along an entire length of a delivery tube). Optionally, the slot may be an opening formed in a side of a delivery tube that does not extend to a distal end of the delivery tube. In some embodiments, the delivery tubes may extend along an entire length of the catheter and may be approximately the same length as the tensioning tube. Optionally, the delivery tubes may not extend along an entire length of the catheter (or tensioning tube), and may be shorter in length than the tensioning tube such that they are disposed at or near a distal end of the catheter.

As shown in FIG. 1, the distal ends 112(b) of the sutures 112 exit the distal end 115(b) of the tensioning tube 115, and are inserted within the delivery tubes 111 via the slots (one suture per delivery tube) to be deployable from the distal ends of the delivery tubes 111. The delivery tubes 111 may be advanced within the delivery catheter 120 (with or without a guidewire) such that the distal end 111(b) of each delivery tube may be positioned proximate a tissue region and/or structure surrounding an opening to be closed for subsequent deployment, desired positioning, and anchorage of the distal ends of the sutures around a periphery of the opening (as discussed below). As such, a suture 112 may be deployed through the lumen 111(a) of each of the plurality of delivery tubes 111, by entering from the slot 111(d) to exit via the distal end of each delivery tube. Similar to the tensioning tube, each of the delivery tubes may be axially rigid.

In some embodiments, delivery tubes vary in size depending on the size of the suture anchor being used. In some examples, a delivery tube has an outer diameter (OD) of about 0.030″, about 0.032″ about 0.033″, about 0.034″, about 0.035″ about 0.037″, about 0.040″, or the like; and an inner diameter (ID) of about 0.023″, about 0.025″, about 0.026″, about 0.027″, about 0.029″, about 0.030″, or the like. In some other examples, the anchor delivery tubes have an OD of about 0.030″-0.040″, about 0.032″-0.038″, about 0.034″-0.036″, or the like; and an ID of about 0.020″-0.030″, about 0.022″-0.028″, about 0.024″-0.026″, or the like. The inner and outer diameter of the delivery tubes can vary depending on the size of the suture anchor disposed within the delivery tube, size of the catheter, or the like.

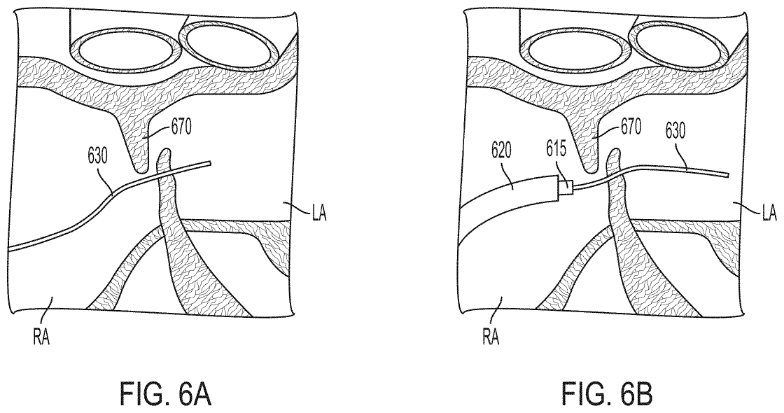

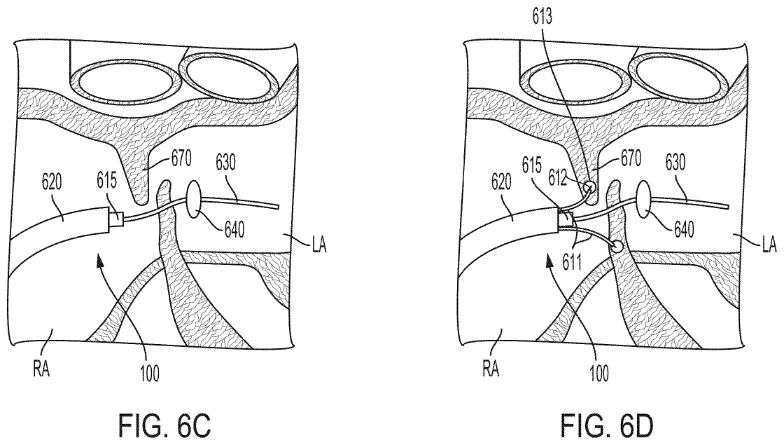

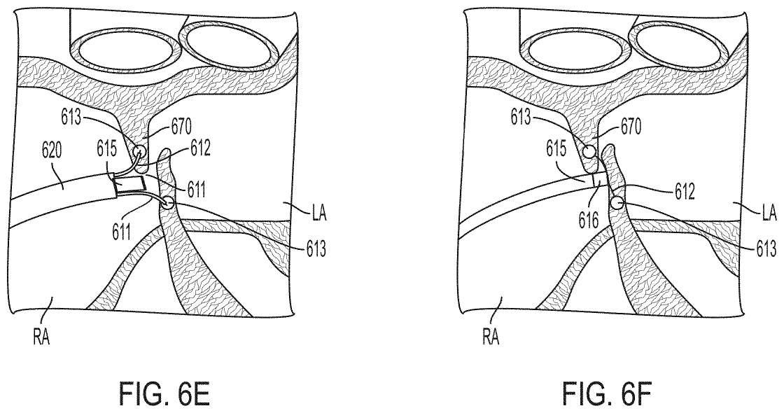

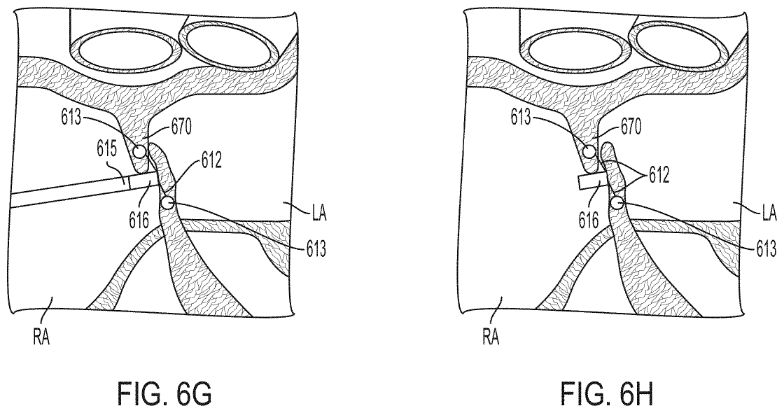

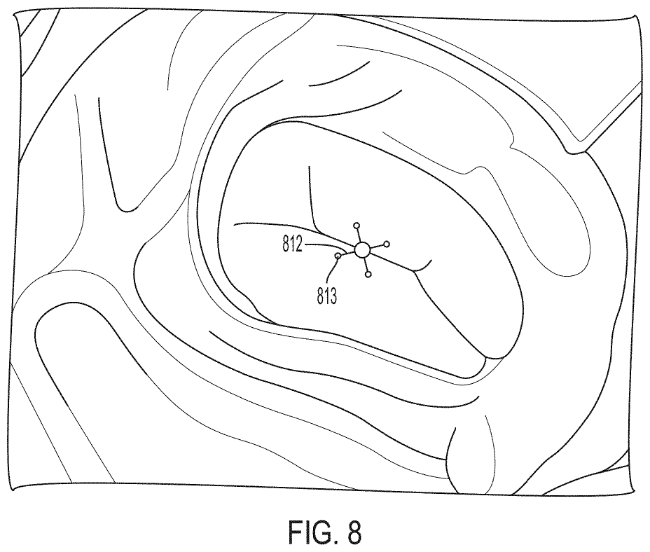

Further, the disclosure illustrates four delivery tubes 111, but this is merely for illustrative purposes. The precise number and spacing of delivery tubes 111 can be left to the particular application. For example, two tubes 111 may be used for placement of two sutures for PFO track opening (as shown in FIGS. 6A-6F). It is anticipated that the larger an opening the larger the number of sutures 112 and hence a larger number of delivery tubes 111 are required. In some applications, the number of sutures may also depend on, for example, force needed to pull together tissue surrounding an opening, a location of the opening, a type of the opening, a shape of the opening, or the like. For example, FIGS. 8A-8I illustrate exemplary embodiments having two, four, and six sutures. Regarding the spacing, the tubes 111 may be evenly (e.g., radially around a guidewire and/or the tensioning tube) and/or unevenly spaced within the lumen of the delivery catheter 120.

The construction of the delivery tubes 111 and the tensioning tube 115 may be from any material that is sufficiently rigid to be steered within a delivery catheter 120, is resistant to deformation in a direction perpendicular to the longitudinal axis of the tube (i.e., axially rigid), but can accommodate bending along the longitudinal axis for steering within the delivery catheter.

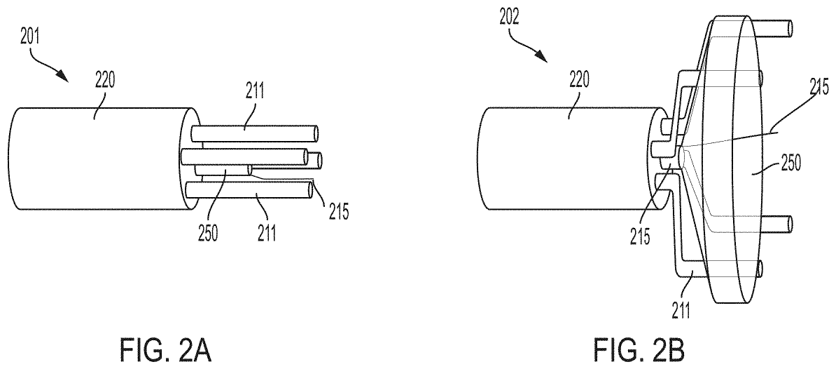

In certain embodiments, a positioning element 250 may be advanced over the tensioning tube and/or the guidewire, between a space defined by the delivery tubes, to cause the delivery tubes 211 to bend or flex outwards from an initial state 201 to a bent state 202 when the positioning element is expanded (as shown in FIG. 2). The positioning element may be separate from or integral with any part of the system 100 and/or device 110. They may be removably or fixedly mounted on the guidewire 130, delivery catheter 120, device 110 (and/or its components) and/or another device. Expansion of positioning element may be selectively controlled to control positioning of the delivery tubes 211 (and hence control the position of the sutures) with respect to the opening before deployment of the suture anchors without increasing the footprint of the delivery catheter or the tensioning tube. The positioning element may be, for example, an inflatable balloon, a disk, a cage, a ball, a mesh, a stent or other structure that is in a first compressed state for advancement over the guidewire to a space between the delivery tubes. and may assume a second expanded state to bend the delivery tubes outwards.

Referring back to FIGS. 1A and 1B, in various embodiments, a distal end of each of the sutures 112 is coupled to a suture anchor 113, each suture anchor being in a collapsed state while being sheathed within a delivery tube. Suture anchors 113 can be deployed in the surface of a tissue (or structure) proximal to the opening to attach or anchor the corresponding suture 112 to the tissue (e.g., at one or more locations near the periphery of an opening). Expansion of the suture anchors may be achieved using, for example, a mechanical expanding feature, inflation of a balloon, or the like; via or within the delivery tubes. Optionally, the suture anchors can be deployed using, for example, a spring-loaded mechanism (e.g., within the delivery tubes) to create a consistent and uniform force for anchor deployment.

Various forms of the suture anchors are described below in more detail. In various embodiments, when the suture anchors 113 reside within the delivery tube 111, they are configured to be in a first compressed form for movement within the delivery tube lumen and for insertion within the tissue. Once inserted or deployed within the tissue, the suture anchors 113 are configured to open or expand, and anchor the distal end of the corresponding sutures to the tissue. It will be understood to those of skill in the art that any now or hereafter known system can be used for deploying the suture anchors around the periphery of a tissue opening.

The suture anchors 113 can be made of super elastic or shape memory nitinol material wire(s). The nitinol wire(s) may be super-elastic and permit the anchors to assume a compressed state for insertion through the vascular wall without catching on or damaging the vascular wall. For example, the nitinol material wire(s) may be preformed by heat treating into a curvature, for example a hook shape, that will act as a suture anchor. The curvature in the wire(s) can be straightened out by drawing into the tube 111. After the suture(s) is advanced out of the tube(s) and the curvature reforms to anchor the suture. Similarly, nitinol tubes may be laser cut into a hook shape and shape-set by heating the cut tube to a certain temperature. Then, the nitinol tubes can be pulled into their original tube shape within the tube, then return to their curved hook shape when removed from the tube upon deployment. Optionally, the anchors may be disposed within removable sheaths that may be removed allowing the anchors to return to their hook shape.

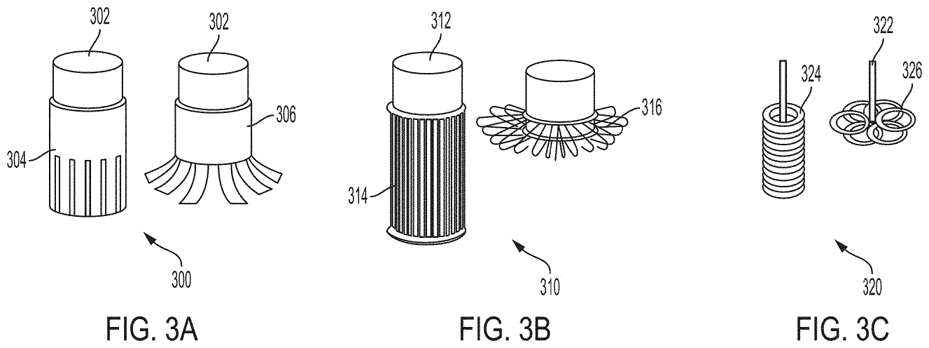

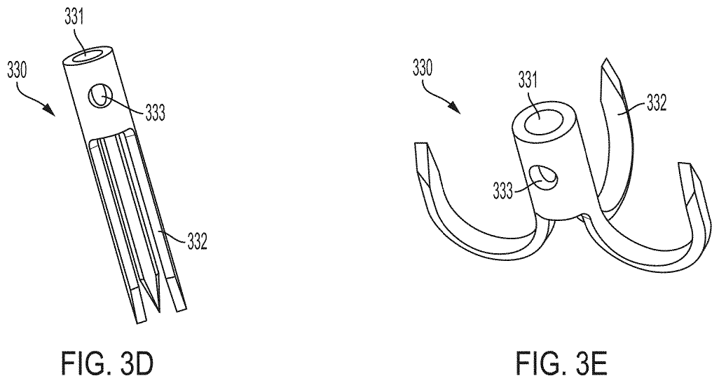

Referring to FIG. 3A-3E, various types of suture anchors are possible, such as tube-based nitinol anchors 300, wire-based nitinol anchors 310 and suture knot anchors 320. Tube-based anchor 300 is illustrated at the end of suture 302. In a compressed or not expanded form 304, the anchor may be about the same diameter or slightly larger than the suture 302. When the anchor 300 is no longer compressed (e.g., within a delivery tube) and is permitted to reach its expanded shape 306 (e.g., when pushed out of a delivery tube), the anchor may have a plurality of hooks or fingers that flare out and get embedded within one or more layers of tissue proximate the opening to be closed.

Similarly, wire-based anchor 310 may have a compressed form 312 (e.g., when it is inside a delivery tube) and in an expanded form 316, have a variety of small wires that spring out embed within the tissue. Suture based anchors 320 (see FIG. 3C) may include a cylindrical knot of suture 324 that is inserted into the tissue. When the length of suture 322 is pulled on to the tension the suture 322, the knot 324 can be pulled into an expanded form 326, which can attach to the tissue. Another example embodiment of a tube-based anchor is illustrated by FIGS. 3D and 3E, which show a three-prong laser cut tube-based anchor. As described above, a nitinol tube may be laser cut into a hook shape and shape-set by heating the cut tube to a certain temperature. Then, the tubes can be pulled into their original tube shape into the delivery tubes of device 110, then return to their curved hook shape when removed from the delivery tubes. Tube-based anchor 330 can include a tube body with a bore 331 running therethrough. Prongs 332 and/or hole 333 may be laser cut into the tube. Hole 333 can be used to attach a suture to tube-based anchor 330, as described above. For example, a knot or reflowed suture ball may prevent a suture run through 331 from pulling back through hole 333. After tube-based anchor 330 is cut, it may heat formed into the hook shape illustrated in FIG. 3E. For example, prongs 330 may be formed into hooks that can engage with tissue and attach to one or more layers of the tissue surface. Tube-based anchors may be compressed (e.g., a shape such as that illustrated by FIG. 3D) inside a deployment device such as a tensioning tube and/or a delivery tube of the current disclosure. When released from the deployment device, tube-based anchor 330 can return to deployed hook shape illustrated in FIG. 3E. While the anchors of FIG. 3 are described as either nitinol or suture-based anchors, anchors may be made from other suitable materials. Such anchors can provide other advantages in addition to ease of anchorage of sutures and relative strength. Though not illustrated in FIG. 3, other types of anchors are also possible, such as pledget-based anchors. In some examples, one or more of the anchors may have additional features to facilitate radial cinching of an opening. For example, a hole (or any other opening) may be provided on a side of an anchor (e.g., 333 in the side of tube based anchor shown in FIGS. 3D and 3E) for suture exit, where a suture exiting from a side (e.g., radially) when tensioned will create a radial or sideway force instead of an outward pulling force.

While the figures illustrate anchor-based sutures, the disclosure is not so limiting and non-anchor-based sutures may similarly be used without deviating from the principles of this disclosure. For example, sutures can be tied with one another and form a knot by going over themselves within tissue to close the opening. In other examples, a suture knot in low profile could be delivered within the tissue and then pulled to form a larger knot that can act as an anchor.

Once the sutures are anchored into tissue proximate to the opening (via the suture anchors), the sutures may be suitably tensioned (e.g., by tensioning or pulling of the proximal ends thereof) to close the opening by radial cinching. As discussed above, the tensioning tube when positioned against (or near) a tissue or structure near the opening cause the sutures to pull the anchored sutures and tissue in towards the opening (instead of pulling of the tissue outwards towards the proximal end of the suture) to cause radial cinching of the opening. Each of the plurality of sutures may be individually deployed, tensioned, advanced and/or otherwise controlled to selectively cinch an opening located between the suture anchors. For example, when four sutures are anchored in an approximate square shape around the periphery of an opening, application of similar tension on each of the four sutures will cause radial cinching of the opening symmetrically (i.e., approximately towards the center of the opening). On the other hand, variable tensioning of the sutures with respect to each other may cause asymmetrical radial cinching of the opening (i.e., offset with respect to the center of the opening).

In various embodiments, the suture(s) 112, can be locked into a desired tensioned state for cinching an opening closed by knotting (or deploying a knot) at a suitable location along the suture length and/or can be locked into a tensioned state using a suture lock. The tails of the suture may then be cut from the knot or suture lock. In yet another alternative embodiment, the tails of the suture may be severed by applying a pre-determined amount of force to the tails of the suture, wherein the tails of the suture will sever due to the applied force. The severance of the suture tails in this embodiment may be further enhanced with the addition of a feature or features formed along the length of the suture thereby forming a weakened zone or region. The suture(s) 112, can be locked into a desired tensioned state when, for example, additional access through the opening is no longer needed, opening needs to be permanently closed, etc.

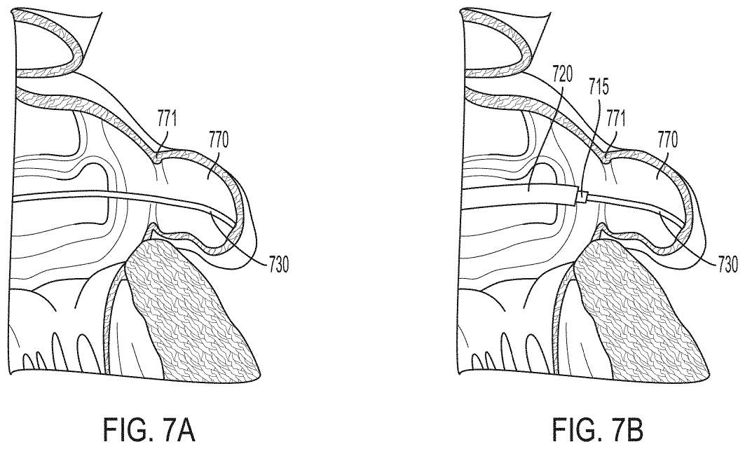

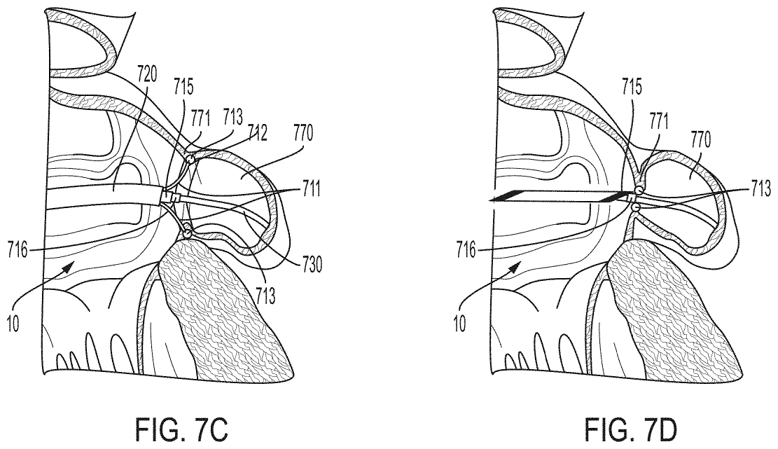

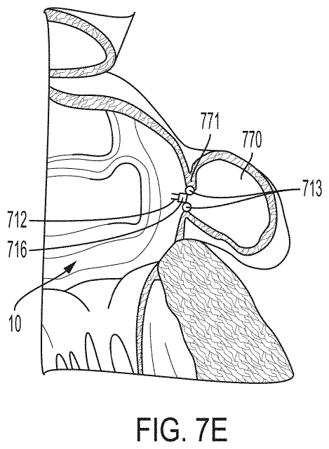

In some embodiments, a suture lock 116 (shown in FIGS. 6 and 7), upon activation, can hold the sutures 112 in tension to keep the opening permanently closed when additional access through the opening is no longer needed. The locking mechanisms (e.g., suture locks) may be separate from or integral with any part of the system 100 or device 110. They may be removably or fixedly mounted on the guidewire 130, delivery catheter 120, device 110 (and/or its components) and/or another device. In some examples, the suture lock 116 is disposed at or near the distal end 115(b) of the tensioning tube 115, and may be advanced over the tensioned sutures to a location between the distal end of the tensioning tube and the opening. The suture lock when advanced may, optionally, provide additional inward radial cinching force on the anchored sutures. Optionally, a suture lock is included and/or advanceable over the tensioning tube 115 and/or the sutures 112 of the device 110 within the delivery catheter lumen and/or over the delivery catheter such that the suture lock can be appropriately positioned over the sutures to be locked.

The suture lock may be a ferrule configured to be crimped around the plurality of sutures. In other embodiments, the suture lock may be a semi-permanent suture lock. For example, the suture lock can be ferrule that is fabricated of a tubular material where the locking pressure is derived by collapsing the tubular walls onto the plurality of sutures. Collapsing the tubular walls permanently distorts the tube to permanently lock the sutures within a lumen of the tube. The tubular material may be advanced by sliding over the plurality of sutures 112 and/or plurality the tubes 111 (together) and can be crimped, causing it to be crushed around and grip the sutures together, holding them in place at a location between the distal ends of the tubes and the opening. In some examples, a separate device is routed over the guidewire 130 and crimps crushes the tubular material together. Other types of suture locks are possible.

Before closure of an opening is performed, it may be desirable to temporarily stabilize the interventional tool in relation to a tissue including the opening and/or proximate the opening. By “stabilization” it is meant that the device 110 will be coupled to a tissue so that any existing relative motion between the device and a structure including the opening is lessened. The stabilization may be terminated after the sutures are appropriately anchored but before closure procedure is completed (e.g., by withdrawal of the stabilization mechanism/tool), but in some instances the stabilization could be terminated and redeployed multiple times at various points throughout the procedure.

The stabilization mechanisms may be separate from or integral with any part of the system 100 or device 110. They may be removably or fixedly mounted on the guidewire 130, delivery catheter 120, device 110 (and/or its components) and/or another device. Likewise, the elements may be components or parts of components of the device which provide one or more additional functions in the opening closure procedure, such as steering, orientation assessment, grasping, coaptation, adjustment or fixation. Further the mechanisms may be such that they may or may not cross the opening in the stabilization process. In particular, such mechanisms may be used to steer and/or orient the components and systems prior to or simultaneous with stabilization.

For example, in a stabilization mechanism may be used to engage and lock the delivery catheter 120 (and/or the device 110) in place in relation to the interatrial septum during a procedure for closure of a PFO track opening. FIG. 6C-6G depicts a stabilizer 550 introduced over a guidewire 530 to cross the PFO track, and form an anchor against the septum to stabilize the system (i.e., positioned on a side of the opening that is opposite the device 110 deployment side). The stabilizer may be, for example, inflatable balloons, disks, cages, balls, mesh, stents or other structures that are in a first compressed state for crossing the PFO septum (or opening being closed) over the guidewire and may assume a second expanded state when it crosses the PFO track. In some examples, the stabilizer is withdrawn from the opening over the guidewire after suture anchors have been deployed but before the opening is closed.

In various embodiments, upon deployment of the suture anchors into tissue and/or structure(s) around the periphery of the opening, only certain components of the system 100 are left within the internal structure while the remaining components are withdrawn before closure of the opening. For example, the delivery catheter, the delivery tubes, and the stabilizer may be withdrawn while the anchored sutures, the tensioning tube, the suture lock and the guidewire may not be withdrawn. After closure of the opening, the tensioning tube and the guidewire may also be withdrawn leaving behind only the anchored sutures held in a tensioned state by the suture lock for keeping the opening radically cinched. As such, a leftover footprint of foreign devices within the biological structure after the closure procedure is minimal compared to prior art methods and devices. This allows for optimal healing of the opening and the biological structure and does not hinder future interventions that require creation of openings or access within the biological structure.

### Methods of Use

The suturing device and system in accordance with the present disclosure may be advanced over a guidewire or deployed using any now or hereafter known methods. Specifically, a tensioning tube (with the sutures disposed within) and a plurality of delivery tubes (with the distal ends of the sutures and anchors) can be advanced proximate a periphery of the opening (specific examples are discussed later) over a guidewire (with or without a delivery catheter). The distal ends of the sutures can then be advanced through each of the delivery tubes, and the suture anchor may be used to anchor the sutures into tissue about a periphery of the opening. The exact location which the sutures and suture anchors are deployed about a periphery of the opening can vary depending on the size, shape, and location of the opening. Optionally, bespoke deployments may be required as openings, cavities, and defects can vary greatly between individuals. In various embodiments, a positioning element may be used for controlling the positioning of the anchors. After deployment of the suture anchors, various components of the system may be withdrawn (e.g., delivery catheter, delivery tubes, positioning element, stabilizer, etc.). Next, the sutures are configured to cause radial cinching of the peripheral tissue to close the opening when the sutures are pulled or otherwise tensioned. Typically, the sutures may be tensioned by manipulating at their respective proximal ends (e.g., pulling, length shortening, etc.) at or near the proximal end of the tensioning tube. As discussed, the tensioning tube prevent movement of the sutures in an axial direction away from the opening and/or outward pulling of the tissue towards the proximal end of the sutures, and instead cause to sutures to apply a radially inward force to cause the peripheral tissue to radially cinch the opening closed. In some examples, the tensioning tube prevents movement away from the opening (e.g., in the distal to proximal direction) because the tensioning tube is axially rigid and positioned to cinch the sutures together, as discussed above. In some examples, the system further includes a suture lock which can permanently lock the sutures in a tensioned state.

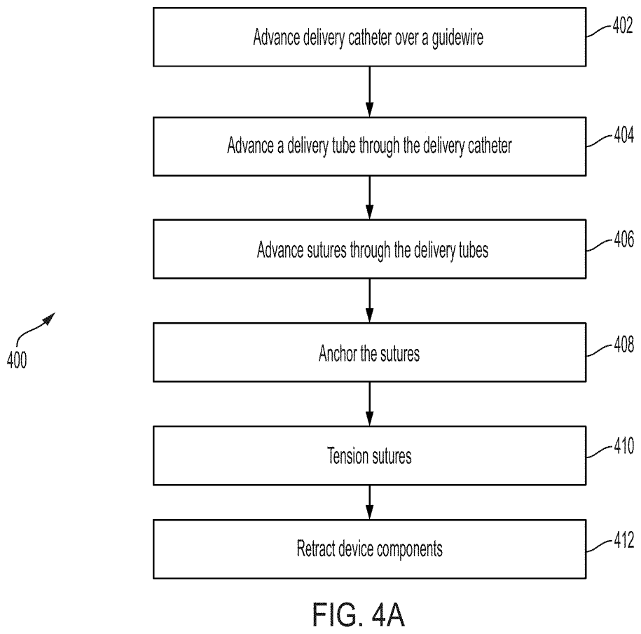

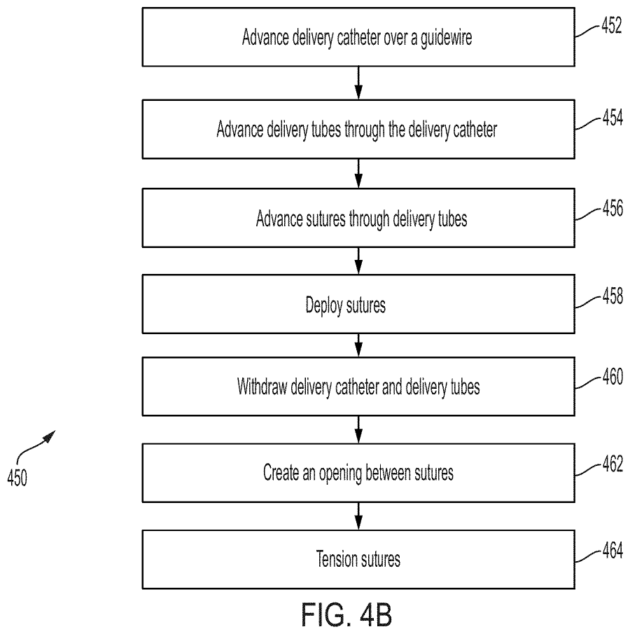

FIG. 4A is a flow chart depicting a method 400 of use for the system of FIG. 1. The method 400 includes a first step 402 of advancing a delivery catheter over a guidewire. The delivery catheter can include a lumen, within which a tensioning tube and a plurality of delivery tubes can be advanced and/or disposed for deployment. As discussed, a plurality of sutures may be deployed from a proximal end of the tensioning tube to exit a distal end thereof, each of the plurality of sutures including a suture anchor at the respective distal end. The distal end of each suture (with the suture anchor) is configured to be inserted within a delivery tube for deployment. In some examples, a stabilizer similar to the stabilizers discussed above can, optionally, be deployed over the guidewire to support an area that is desired to be cinched.

The method additionally includes a step 404 of advancing the plurality of delivery tubes through the delivery catheter towards tissue or a structure proximate an opening. Optionally, an arrangement including the tensioning tube and the plurality of delivery tubes with the sutures disposed may be simultaneously or near simultaneously advanced towards an opening within the lumen of the delivery catheter. Next, the method includes advancing sutures through each of the delivery tubes (406) and anchoring the sutures within the tissue or structure (408) using, for example, suture anchors discussed above. Optionally, the positioning of the anchor sutures may be controlled using a positioning element. The catheter and the delivery tubes (including the stabilizer, and the positioning element) may be withdrawn.

The sutures can then be tensioned (while being held together by the tensioning tube) to radially cinch the peripheral tissue of an opening inwards to close the opening (410). Optionally, the sutures can then be locked in a tensioned state using a suture lock and/or knot for permanently closing an opening. Optionally, the tail ends of the sutures may be cut, and the remaining components of the system (e.g., tensioning tube and the guidewire) may be withdrawn.

FIG. 4B is a different flow chart illustrating a method 450 of use of the system of FIG. 1 when an opening is created during a procedure (instead of when an opening already exists). For example, an access hole may need to be created in the septal wall between the left atrium and a right atrium to perform a procedure within the left atrium (e.g. mitral valve repair/replacement, left atrial appendage occlusion etc). The method includes a first step 452 of advancing a delivery catheter over a guidewire. The catheter is advanced proximate a site where an opening is to be created for a procedure. As discussed, the delivery catheter can include a lumen, within which a tensioning tube and a plurality of delivery tubes can be advanced and/or disposed for deployment. A plurality of sutures may be advanced from a proximal end of the tensioning tube to exit a distal end thereof, each of the plurality of sutures including a suture anchor at the respective distal end. The distal end of each suture (with the suture anchor) is configured to be inserted within a delivery tube for deployment. Optionally, an arrangement including the tensioning tube and the plurality of delivery tubes with the sutures disposed may be simultaneously or near simultaneously deployed towards an opening within the lumen of the delivery catheter.

The method additionally includes a step 454 of advancing the plurality of delivery tubes through the delivery catheter, deploying sutures through each of the delivery tubes (456) and anchoring the sutures within the tissue or structure (458) using, for example, suture anchors discussed above. The anchors may be positioned to at least partially surround an area within which the access or opening will be created. Optionally, the positioning of the anchors may be controlled using a positioning element. At 460, the delivery catheter and the delivery tubes (including the stabilizer, and the positioning element) may be withdrawn.

At 462, tools (e.g., another catheter) for performing one or more steps of the procedure may be advanced over the guidewire. For example, an opening may be created at least partially between the deployed anchors using the tools, and a procedure may be performed. Optionally, the sutures may be suitably tensioned so as to keep the opening at least partially cinched over the procedural tool during the procedure while the procedural tool is inserted within the opening.

Upon completion of the procedure, the tools may be withdrawn, and the sutures can be tensioned (while being held together by the tensioning tube) to radially cinch the peripheral tissue of the created opening inwards to close the opening (464). Additionally, the sutures can then be locked in a tensioned state using a suture lock and/or knot for permanently closing an opening. Optionally, the tail ends of the sutures may be cut, and the remaining components of the system (e.g., tensioning tube and the guidewire) may be withdrawn. Kits:

Kits for closure of openings in an internal biological structure are also provided. In general, the kits comprise a suturing device including sutures, a tensioning tube, and a plurality of delivery tubes. The sutures may be disposed within the tensioning tube from a proximal end of the tensioning tube to exit a distal end of the tensioning tube. The sutures may have a proximal end and a distal end, with optional anchors attached to the sutures at its distal end and configured to secure the suture to the periphery of an opening. The distal ends of the sutures may be disposed within the delivery tubes.

A catheter for delivery of the suturing device to the proximity of the opening may be included in the kits. Additional catheters may be included in the kits as well. Optionally, a guidewire may be included in the kits.

Any number of appropriate catheters may be used with devices and methods described herein, and may be included as part of a kit. For example, individual catheters may be used to perform a procedure (with or without creation of an opening), deliver and/or implant the stabilizer, to delivery and/or deploy the positioning element, to deliver and/or secure a suture lock, or the like. Additional catheters may also be included. In some variations, catheters may be combined.

In addition, as evidenced by the description of the devices and methods above, it may be advantageous to provide a kit with additional tools useful in carrying out the described methods. For example, the kits may further comprise a cutting wire for cutting the sutures, a stabilizer, a positioning element, and/or a suture lock.

The kit may also include instructions on how to use the contents of the kit. For example, Optionally, the kits may include instructions for advancing the sutures within the tensioning tube and/or the delivery tubes. Instructions may include reference materials (including indications for use, etc.) and be in any appropriate format, including written, pictographic, visual, electronic, etc., and be in any language, or multiple languages.

### EXAMPLES

A human heart has four chambers. The upper chambers are called the left and right atria, and the lower chambers are called the left and right ventricles. A wall of muscle called the septum separates the left and right atria and the left and right ventricles. That portion of the septum that separates the two upper chambers (the right and left atria) of the heart is termed the atrial (or interatrial) septum while the portion of the septum that lies between the two lower chambers (the right and left ventricles) of the heart is called the ventricular (or interventricular) septum.

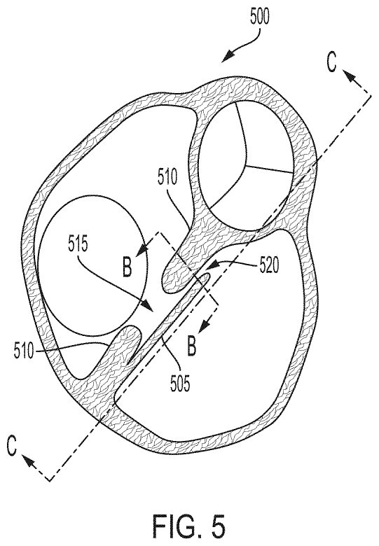

FIG. 5 illustrates a short-axis view of the heart 500 at the level of the right atrium (RA) and left atrium (LA), in a plane generally parallel to the atrio-ventricular groove, and at the level of the aortic valve. This view is looking from caudal to cranial. FIG. 5 also shows the septum primum (SP) 505, a flap-like structure, which normally covers the foramen ovale 515, an opening in the septum secundum (SS) 510 of the heart 500. In utero, the foramen ovale 515 serves as a physiologic conduit for right-to-left shunting of blood in the fetal heart. After birth, with the establishment of pulmonary circulation, the increased left atrial blood flow and pressure presses the septum primum (SP) 505 against the walls of the septum secundum (SS) 510, covering the foramen ovale 515 and resulting in functional closure of the foramen ovale 515. This closure is usually followed by anatomical closure of the foramen ovale 515 due to fusion of the septum primum (SP) 505 to the septum secundum (SS) 510.

The PFO results when either partial or no fusion of the septum primum 505 to the septum secundum 510 occurs. When this condition exists, a passageway (PFO track) 520 between the septum primum 505 and septum secundum 510 may allow communication of blood between the atria. This PFO track 520 is typically parallel to the plane of the septum primum 505, and has an opening that is generally oval in shape. Typically, the septum primum 505 acts like a one-way valve, preventing fluid communication between the right and left atria through the PFO track 520. Occasionally, the pressure in the right atrium may temporarily be higher than the left atrium. When this condition occurs, the PFO track 520 opens and allow some fluid to pass from the right atrium to the left atrium. In other cases, although the PFO track 520 is often held closed, the endothelialized surfaces of the tissues forming the PFO track 520 can prevent the tissue from healing together and permanently closing the PFO track 520. The present invention relates to a system and method for closing a passageway in a body. In a particular embodiment, the device is used to close the Patent Foramen Ovale in a human heart. One of ordinary skill in the art would understand that similar embodiments could be used to close other passageways and openings in the body without departing from the general intent or teachings of the present invention.

A procedure for closing a PFO using a system of FIG. 5 in accordance with the principles of the disclosure is now described with respect to FIGS. 6A-6H. Subcutaneous access to the right common femoral vein may be gained via a puncture in the groin area of a patient. For example, a trans-septal sheath or similar sheath (e.g., a delivery catheter) is advanced through the puncture, into the femoral vein and further through the inferior vena cava. The sheath may be further advanced into the right atrium of the heart. It will be appreciated that other routes known by those skilled in the arts can additionally be used to route the sheath. After placing the sheath in a desired location, a guidewire may be advanced through the sheath and positioned appropriately relative to the guidewire for the duration of the procedure. Alternatively, the guidewire may be advanced towards the PFO without the use of a sheath or delivery catheter, and the delivery catheter may be advanced using an already deployed guidewire (as discussed below with respect to FIGS. 7A-7F. The suturing device in accordance with the present invention may then be advanced over the guidewire. Proper placement of the device may be determined through the use of imaging technologies such as X-ray or fluoroscopy.

For example, FIG. 6A depicts a tensioning tube 620 (similar to 120) being routed to a right atrium RA of a heart (i.e., near a PFO opening). FIG. 6B depicts a guidewire 630 (similar to 130) being extended within the delivery catheter lumen into the left atrium LA through the PFO opening 670. Optionally, in some examples, the guidewire may cause a transseptal puncture through the septum wall as the guidewire 130 is advanced into the left atrium LA. FIG. 6C depicts a stabilizer 640 being deployed into the left atrium and expanded to stabilize the system with respect to the PFO opening 670. Once the system is stabilized, sutures disposed within a tensioning tube 615 are deployed via two delivery tubes 611 (similar to 111). Specifically, the delivery tubes 611 are deployed proximate the PFO opening 670 (e.g., near the septal walls in the right atrium RA) as depicted in FIG. 6D. Next, as depicted in FIG. 6E, suture anchors 613 are deployed through the tubes 611 into suitable positions within peripheral tissue or structure surrounding the opening. For example, as shown in FIG. 6E, two suture anchors are deployed—one on each side of the PFO opening in the septum wall to anchor the sutures 612 in appropriate positions with respect to the PFO opening. The stabilizer 640 is then collapsed and removed. Optionally, the delivery catheter and the delivery tubes are also withdrawn. FIG. 6F depicts tensioning of the sutures 612 to cause radial cinching when the tensioning tube 615 prevents movement of the anchored sutures 612 away from each other to cinch the peripheral tissue of the septum wall to close the PFO opening 670. Optionally, a suture lock 616 is disposed at a distal end of the tensioning tube 615 and is advanced over the sutures 612 to lock the sutures 612 in a tensioned state (as shown in FIG. 6G). Finally, in FIG. 6H the tensioning tube 615 and the guidewire 620 are removed from the right atrium and the sutures 612, suture anchors 613, and, optionally, suture lock 616 are left in place to keep the opening closed. The septal defect is then allowed to heal. As discussed above, in some examples, the sutures 612, suture anchors 613 and suture lock 616 are absorbable within the body.

FIGS. 7A-7E depict the system of FIG. 1 being used to close a left atrial appendage (LAA) in accordance with the principles of the present disclosure. It has been estimated that approximately 75,000 atrial fibrillation patients each year suffer a stroke related to that condition. It appears that strokes in these patients result from emboli many of which may originate from the left atrial appendage. The irregular heartbeat causes blood to pool in the left atrial appendage, allowing clots to accumulate over time. From time to time, clot may dislodge from the left atrial appendage and may enter the cranial circulation causing a stroke, the coronary circulation causing a myocardial infarction, the peripheral circulation causing limb ischemia, as well as other vascular beds. The methods and systems of the current disclosure may be used for closing by cinching base region 771 of the LAA 770 shown in FIGS. 7A-7E. By closing off the base region 771, the exchange of materials between the LAA and the left atrium will be stopped. Thus, the release of emboli from the left atrial appendage into the left atrium will be stopped.

As discussed above, access to the base region 771 may be gained intravascularly. FIG. 7A depicts the guidewire 730 (similar to 130) being extended into the LAA 770. A delivery catheter 720 (similar to 120) may then be advanced over the guidewire 730 towards the LAA 770 (shown in FIG. 7B) up to a desired distance away from the base region 771 (e.g., a distance sufficient to deploy the tensioning tube 715 and the delivery tubes 711). The delivery tubes 711 are deployed proximate tissue surrounding the base region 771 in the left atrium LA as shown in FIG. 7C. Additionally, a suture 712 is deployed via the lumen of each delivery tube 711 and anchored via the anchors 713 into the peripheral tissue of the base region 771. Optionally, the delivery catheter and the delivery tubes are also withdrawn. FIG. 7D depicts the sutures 712 being tensioned, while the tensioning tube 715 prevents movement of the sutures 712 away from each other, to cause radially inward cinching the peripheral tissue of the base region 771 for closing the base region 771. Optionally, suture lock 716 is deployed to lock the sutures 712 in a tensioned state. Finally, as depicted in FIG. 7E, the tensioning tube 715 and/or the guidewire 730 is removed from the right atrium and the sutures 712, suture anchors 713 and suture locks 716 are left in place to keep the base region 771 closed.

FIG. 8 depicts an example of sutures 812 (anchored via anchors 813) deployed using the devices and methods discussed above, and tensioned to radially cinch together mitral valve leaflets during a trans-catheter edge to edge repair (TEER) procedure. For simplicity, only deployed sutures and suture anchors are shown. However, it will be appreciated that the sutures 812 and suture anchors 813 can be deployed as discussed above. In some examples, the delivery catheter for TEER is routed through the transfemoral route. However, it will be appreciated that the delivery catheter can also be routed through a trans-naval route, a trans carotid route, a trans atrial route or any other similar route.

It will be appreciated that although closing a left atrial appendage, trans catheter edge-to-edge repair and closing a PFO defect are the only procedures depicted, the disclosure is not so limiting. The devices, systems and methods of the current disclosure also are suitable for closing other openings or passageways, including other such openings in the heart, for example atrial septal defects, ventricular septal defects, and patent ducts arterioses, as well as openings or passageways in other portions of a body such as an arteriovenous fistula, opening in the gastric tract or the like In other examples, the devices, systems, and methods of the current disclosure can be used where a hole is created as a result of another procedure such as transseptal access for transcatheter mitral valve replacement (TMVR). It will also be appreciated that in some examples, such as TMVR, when future access is required, the suture lock can include a feature that allows unlocking and releases tension on the sutures to un-cinch and allow further access to the site. For example, U.S. Pat. No. 11,382,609, which is incorporated entirely by reference herein, depicts a system for dynamic vascular access and describes various suture locks that can be used within the present system.

The above detailed descriptions of embodiments of the technology and examples are not intended to be exhaustive or to limit the technology to the precise form disclosed above. The devices disclosed herein can provide cinching that causes a mechanical closing or minimizing orifices. The mechanical cinching can cause the adherent surface, including skin, to be drawn inwardly towards an orifice to enhance diminution or stoppage of material flow through the orifice. When an instrument is positioned in the orifice, the cinching can push tissue surrounding the instrument towards the instrument, thereby closing (e.g., partially or completely closing) the orifice. The tension in the tensioner can be selectively increased or decreased to increase or decrease the cinching force applied to the subject's skin. The mechanical cinching may also allow for the occlusion and/or tamponade of the orifice. The size of the device can be selected based on the size and location of the orifice in the subject, the configuration of the instrument, if any, positioned in the orifice, and desired functionality. The desired functionality can include closing the orifice, closing or eliminating a gap between the instrument and the subject's skin, tamponading, or the like. Additional features can be coupled to or incorporate into the cinching device so as to impart additional functionality.

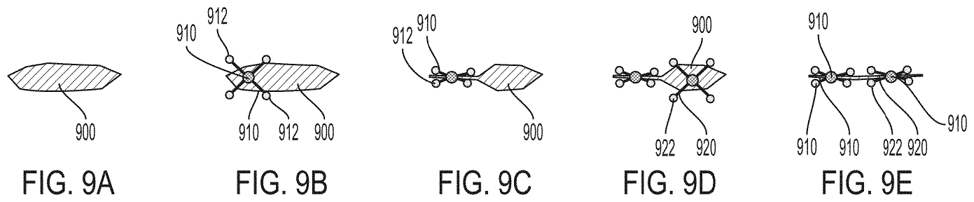

FIGS. 9A to 9E depict an example closure of an opening 900 (FIG. 9A) using the system of FIGS. 1A-1B using two or more sets of sutures (e.g., when the opening is large, irregularly shaped, or the like). For simplicity, only deployed sutures and suture anchors are shown. However, it will be appreciated that the sutures and suture anchors can be deployed as discussed above. FIG. 9B shows a first set sutures 910 deployed on a first side of the opening 900 and anchored using suture anchors 912. FIG. 9C depicts the first set sutures 910 being tensioned to cinch the peripheral tissue around the first side of the opening 900 to close a portion of the opening 900. FIG. 9D shows a first set sutures 920 deployed on a second side of the opening 900 and anchored using suture anchors 922. FIG. 9E depicts the second set sutures 920 being tensioned to cinch the peripheral tissue around the second side of the opening 900 to close another portion of the opening 900. It will be appreciated that any number of sets of sutures can be deployed to close larger or smaller orifices.

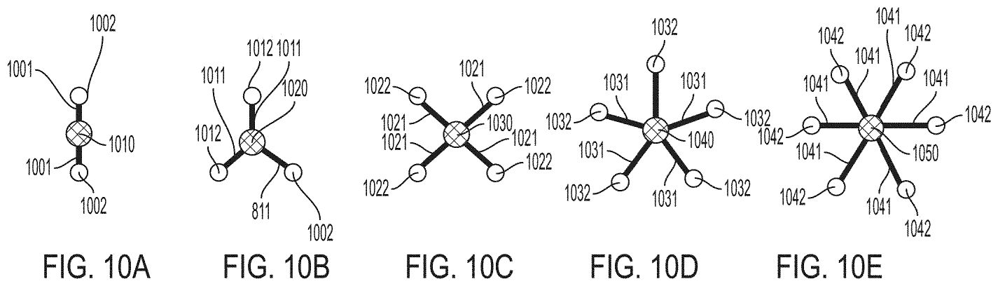

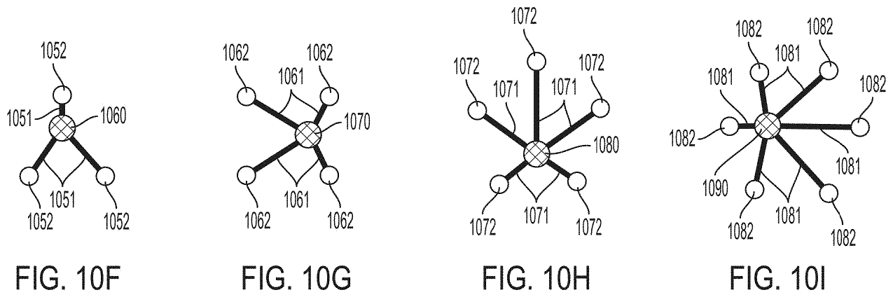

Further, it will be appreciated that although four sutures and four suture anchors are depicted in FIGS. 9B to 9E, and FIGS. 1A-1B depict a sectional view of the system deploying four sutures, the system of FIGS. 1A-1B can be configured to simultaneously deploy any desired number of sutures (e.g., 2, 3, 4, 5, 6, or the like). For example, FIG. 10A depicts two sutures 1001 and two suture anchors 1002 disposed radially around an opening 1010. FIG. 10B shows three sutures 1011 and three suture anchors 1012 disposed radially around an opening 1020. FIG. 10C depicts four sutures 1021 and four suture anchors 1022 disposed radially around an opening 1030. FIG. 10D depicts five sutures 1031 and five suture anchors 1032 disposed radially about an opening 1040. FIG. 10E depicts six sutures 1041 and six suture anchors 1042 disposed radially about an opening 1050. It will also be appreciated that although the sutures and suture anchors are disposed radially and symmetrically around and opening in FIGS. 10A-10E, the disclosure is not so limiting. Rather, sutures and suture anchors can be asymmetrically disposed around an opening in any desired location as shown in FIGS. 10F-10I. FIG. 10F shows three sutures 1051 and three suture anchors 1052 disposed asymmetrically around an opening 1060. FIG. 10G depicts four sutures 1061 and four suture anchors 1062 disposed asymmetrically around an opening 1070. FIG. 10H depicts five sutures 1071 and five suture anchors 1072 disposed asymmetrically about an opening 1080. FIG. 10I depicts six sutures 1081 and six suture anchors 1082 disposed radially about an opening 1090. Furthermore, as discussed above asymmetrical closure may be achieved using individually variable tensioning of the sutures even when the sutures are symmetrically disposed about an opening (or vice versa).

It will be understood that terms such as “same,” “equal,” “planar,” or “coplanar,” as used herein when referring to orientation, layout, location, shapes, sizes, amounts, or other measures do not necessarily mean an exactly identical orientation, layout, location, shape, size, amount, or other measure, but are intended to encompass nearly identical orientation, layout, location, shapes, sizes, amounts, or other measures within acceptable variations that may occur, for example, due to manufacturing processes. The term “substantially” may be used herein to emphasize this meaning, unless the context or other statements clearly indicate otherwise. For example, items described as “substantially the same,” “substantially equal,” or “substantially planar,” may be exactly the same, equal, or planar, or may be the same, equal, or planar within acceptable variations that may occur, for example, due to manufacturing processes and/or tolerances. The term “substantially” may be used to encompass this meaning, especially when such variations do not materially alter functionality.

It will be understood that various modifications may be made to the embodiments disclosed herein. Likewise, the above disclosed methods may be performed according to an alternate sequence. Therefore, the above description should not be construed as limiting, but merely as exemplification of the various embodiments. Those skilled in the art will envision other modifications within the scope and spirit of the claims appended hereto.

What is claimed is:

1. A system for radial cinching of an opening in an internal biological structure, the system comprising:
a tensioning tube extending between a distal end and a proximal end;
a plurality of sutures configured for deployment within the tensioning tube from the proximal end to exit via the distal end of the tensioning tube, wherein the tensioning tube is configured to constrain movement of portions of the plurality of sutures disposed within the tensioning tube away from each other; and
a plurality of delivery tubes, each of the plurality of delivery tubes configured to receive a distal end of one the plurality of sutures after exiting from the distal end of the tensioning tube, wherein the plurality of delivery tubes are further configured to deploy the distal ends of the plurality of sutures proximate a periphery of the opening; and
wherein, upon deployment of the sutures proximate the periphery of the opening, tensioning of one or more of the plurality of sutures while movement of portions of the plurality of sutures is constrained by the tensioning tube causes radial cinching of the periphery to close the opening.

2. The system of claim 1, further comprising a delivery catheter, wherein the tensioning tube and the plurality of tubes are deployable within a lumen of the delivery catheter.

3. The system of claim 2, further comprising a guidewire.

4. The system of claim 3, further comprising a stabilizer deployable over the guidewire and configured to be disposed on a side of the opening that does not include the plurality of sutures such that the stabilizer can stabilize the plurality of tubes with respect to the opening during deployment of the sutures.

5. The system of claim 4, wherein the stabilizer is selected from at least one of the following: a balloon, a stent, a disc, or a sheath.

6. The system of claim 3, further comprising a positioning element deployable over the guidewire and configured to control positioning of the sutures during deployment by causing outward bending of one or more of the plurality of delivery tubes.

7. The system of claim 6, wherein the positioning element is selected from at least one of the following: a balloon, a stent, a disc, or a sheath.

8. The system of claim 6, wherein the plurality of sutures are configured to be either symmetrically deployed or asymmetrically deployed around the periphery of the opening.

9. The system of claim 1, further comprising a suture lock configured to lock the plurality of sutures in a tensioned state after the opening is closed.

10. The system of claim 1, wherein the distal ends of each of the plurality of sutures is associated with a suture anchor, the suture anchor being in a compressed state when the distal end of the corresponding suture is disposed within a delivery tube.

11. The system of claim 1, wherein the suture anchor is configured to be in an expanded state upon deployment from the delivery tube and anchor the corresponding suture to the periphery.

12. The system of claim 1, wherein each of the plurality of sutures is configured to be individually controlled for deployment and tensioning.

13. The system of claim 1 wherein the opening is located within a cardiovascular region.

14. The system of claim 1, wherein the plurality of sutures are made of a material that is absorbable by a human body.

15. A system for radial cinching of an opening in an internal biological structure, the system comprising:
a guidewire;
a delivery catheter; and
a suturing device comprising:
an axially rigid tensioning tube deployable, via the delivery catheter, to the opening;
a plurality of sutures configured for deployment within the tensioning tube from a proximal end of the tensioning tube to exit via a distal end of the tensioning tube, wherein the tensioning tube is configured to constrain movement of portions of the plurality of sutures disposed within the tensioning tube away from each other; and
a plurality of delivery tubes disposed within the delivery catheter, each of the plurality of delivery tubes configured to receive a distal end of one the plurality of sutures after exiting from the distal end of the tensioning tube, wherein the plurality of delivery tubes are further configured to deploy the distal ends of the plurality of sutures proximate a periphery of the opening, the distal ends of the plurality of sutures associated with suture anchors;
a suture lock disposed near the distal end of the tensioning tube; and
wherein, upon deployment of the sutures proximate the periphery of the opening, tensioning of one or more of the plurality of sutures while movement of portions of the plurality of sutures is constrained by the tensioning tube causes radial cinching of the periphery to close the opening.

16. The system of claim 15, further comprising a stabilizer deployable over the guidewire and configured to stabilize the suturing device with respect to the opening during deployment of the sutures.

17. The system of claim 15, further comprising a positioning element deployable over the guidewire and configured to control positioning of the sutures during deployment by causing outward bending of one or more of the plurality of delivery tubes.

18. The system of claim 15, wherein each of the plurality of delivery tubes comprises a slot extending, from a distal end thereof, at least partially along a length of that delivery tube, the slot being configured to receive the distal end of that one the plurality of sutures after exiting from the distal end of the tensioning tube.

19. The system of claim 15, wherein each of the plurality of delivery tubes comprises a mechanism for deployment, by pushing, of a suture from a distal end of that delivery tube.

20. The system of claim 15, wherein the suture lock is configured to be advanced over the plurality of sutures and to lock the plurality of sutures in a tensioned state.

21. A method of closing an opening within an internal biological structure, the method comprising:
deploying a delivery catheter over a guidewire towards the opening;
deploying a tensioning tube within a lumen of the delivery catheter, the tensioning tube being axially rigid;
deploying a plurality of sutures through a proximal end of the tensioning tube to exit from a distal end of the tensioning tube, the plurality of sutures each including a suture anchor at a distal end of the suture;
deploying a plurality of delivery tubes through the lumen of the delivery catheter, distal ends of the each of the plurality of sutures being disposed within one of the plurality of delivery tubes such that the suture anchors are in a compressed state;
anchoring, using the suture anchors, the sutures into peripheral tissue around the opening by pushing the sutures from distal ends of each of the plurality of delivery tubes; and
tensioning the plurality of sutures while movement of portions of the plurality of sutures is constrained by the tensioning tube to cause radial cinching of the periphery to close the opening.

22. The method of claim 21, further comprising deploying a suture lock and activating the suture lock for locking the sutures in a tensioned state after radial cinching of the periphery.

23. The method of claim 21, further comprising deploying an expandable stabilizing device through the lumen of the delivery catheter for anchoring the tensioning tube and the plurality of delivery tubes with respect to the opening.

24. The method of claim 23, further comprising expanding the expandable stabilizing device after deploying the stabilizing device.

25. The method of claim 21, further comprising:
deploying a positioning element through the lumen of the delivery catheter; and
causing, using the positioning element, outward bending of the plurality of delivery tubes for controlling a position of one or more of the suture anchors.

26. The method of claim 21, further comprising withdrawing, before tensioning the plurality of sutures, the delivery catheter and the plurality of delivery tubes.

27. The method of claim 21, further comprising withdrawing, after tensioning the plurality of sutures, the tensioning tube and the guidewire.

28. A method of performing a procedure within an internal biological structure, the method comprising:
deploying a delivery catheter over a guidewire towards the internal biological structure;
deploying a tensioning tube within a lumen of the delivery catheter, the tensioning tube being axially rigid;
deploying a plurality of sutures through a proximal end of the tensioning tube to exit from a distal end of the tensioning tube, the plurality of sutures each including a suture anchor at a distal end of the suture;
deploying a plurality of delivery tubes through the lumen of the delivery catheter, distal ends of the each of the plurality of sutures being disposed within one of the plurality of delivery tubes such that the suture anchors are in a compressed state;
anchoring, using the suture anchors, the sutures into peripheral tissue around the opening by pushing the sutures from distal ends of each of the plurality of delivery tubes;
withdrawing the delivery catheter;
deploying a procedure tool for creating an opening in an area between the anchored sutures;
performing, via the opening, the procedure; and
tensioning the plurality of sutures while movement of portions of the plurality of sutures is constrained by the tensioning tube to cause radial cinching of the periphery to close the opening.

29. The method of claim 28, further comprising withdrawing the procedure tool before tensioning the plurality of sutures.

30. The method of claim 28, wherein tensioning the sutures further comprises tensioning the sutures during performing of the procedure to keep the opening at least partially cinched over the procedure tool.
